(19) United States
(12) Patent Application Publication
(10) Pub. No.: US 2005/0085905 A1
(43) Pub. Date: Apr. 21, 2005

(54) Trabeculectomy (guarded filtration procedure) with tissue re-enforcement

(76) Inventor: Asher Weiner, W Bloomfield, MI (US)

Correspondence Address: MORGAN & FINNEGAN, L.L.P., 3 WORLD FINANCIAL CENTER, NEW YORK, NY, 10281-2101, US

(21) Appl. No.: 10/497,761

(22) PCT Filed: Dec. 6, 2002
(86) PCT No.: PCT/US02/39304

Related U.S. Application Data
(60) Provisional application No. 60/338,432, filed on Dec. 6, 2001.

(51) Int. Cl. A61F 2/14

(52) U.S. Cl. 623/4.1; 623/905

(57) ABSTRACT

An ocular implant (10) is disclosed for beneficially inhibiting wound healing, inflammation, and devastating infection following a guarded filtration procedure. The implant (10) is comprised of a thin implantable material contoured to fit the eye. During a guarded filtration procedure, the implant (10) is positioned at the edge of the sclerectomy site, under the scleral flap (7), and extends laterally and posteriorly from the sclerectomy site. The implant (10) significantly inhibits adhesion and scarring at the surgical site, and eliminates the need for anti-scarring medications, thereby reducing the risk of blinding infections.

### TECHNICAL FIELD OF THE INVENTION

The present invention relates to ocular implants, and, more specifically, to improved methods and devices useful in performing a trabeculectomy or guarded filtration procedure (GFP), that beneficially inhibit wound healing, inflammation, infection, and scarring following a guarded filtration procedure.

### BACKGROUND OF THE INVENTION

Glaucoma is a progressive eye disease, which affects millions of adults each year. If left untreated, glaucoma causes partial or total blindness, and is among the leading causes of blindness in all countries. Glaucoma occurs when the pressure inside the eye rises above safe levels due to poor drainage or blockage of the aqueous (the fluid produced inside the eye) outflow channel, or due to increases in venous pressure outside of the eye. The increased intraocular pressure damages the tissues in the eye, especially the optic nerve, which eventually causes blindness.

One method for treating progressing glaucoma is a trabeculectomy (also referred to as guarded filtration procedure (GFP)). In traditional guarded filtration surgery, the sclera is exposed, and a scleral flap is dissected in the scleral tissue. The scleral flap is elevated and pulled forward to reveal a bed of scleral tissue under the flap. An incision (referred to as a sclerectomy) is made through the scleral bed to create a “window” or fistula into the anterior chamber of the eye, which allows the aqueous (the fluid produced in the eye) to flow out of the anterior chamber, thereby alleviating the intraocular pressure. The scleral flap is sutured over the fistula, creating a small space under the flap which allows the aqueous to drain from the eye, yet provides enough resistance so that excess aqueous does not escape, thus reducing the risk of hypotony.

A major problem with filtration surgery in general is the eye's own natural wound healing response, which causes the fistula to close or otherwise heal too rapidly, which, in turn, causes the filtration (i.e. drainage) to fail. Attempts to overcome this problem have included inserting ophthalmic devices such as tubes, valves, or shunts into the fistula in order to maintain the fistula open. These conventional drainage devices have been widely used with varying degrees of success. Examples of such devices are disclosed in U.S. Pat. Nos. 5,178,604; 5,397,300; 5,868,697; 5,879,319. However, these implants often become clogged, obstructed, or restricted by the proliferation of scar tissue and adherence of the tissue layers, which occur at the surgical site. Most of these implant devices can also cause restriction of eye movement, incapacitating double vision, and eye discomfort. In addition, to counteract the natural healing process and closing of the fistula in filtration surgery, antimetabolite drugs are commonly used in filtration surgery to inhibit the wound healing process. Unfortunately, a major complication of using antimetabolites is that they weaken and thin healthy tissues, increasing the risk of developing a blinding infection by nearly ten-fold.

U.S. Pat. Nos. 4,634,418 and 6,102,045 disclose other types of drainage devices that are constructed of absorptive material that act as wicks or absorb the aqueous which drains from the anterior chamber of the eye to the area beneath the scleral flap. However, these devices do not address the scarring around the scleral flap, which causes guarded filtration procedures to fail.

It is thus one object of the present invention to provide a method and implant for beneficially inhibiting wound healing in the eye that would otherwise cause unwanted closure of the surgical fistula.

It is another object of the present invention to provide an ophthalmic implant made of an appropriate biocompatible material, and of an appropriate size and shape to effectively inhibit unwanted wound healing in the eye following filtration surgery.

It is yet a further object of the present invention to provide a simple method for positioning the implant during a guarded filtration procedure.

### BRIEF SUMMARY OF THE INVENTION

The present invention relates to trabeculectomy (GFP) with tissue re-enforcement that avoids the need to use antimetabolites or other agents often employed for improving the success rate of glaucoma filtration surgery, while reducing the rate of devastating infection and other complications. The procedure involves the placement of an implant, which acts as a mechanical barrier between the tissues that tend to scar and adhere to one another at the surgical site. Thus, the implant improves the long-term success rate of the filtration procedure, and diminishes post-operative infection rates. The implant is made of implantable-grade material of approximately 7 mm×10 mm dimensions, and is contoured to conform to the globe of the eye.

The method of the present invention, in a preferred embodiment, involves the steps of exposing the sclera, dissecting a scleral flap, performing a sclerectomy, and then securing a thin, implantable-grade biocompatible material such as silicone, methylmetacrylate, or another material to which tissues do not adhere (from henceforth to be referred to as the “implant”), to the posterior sclerectomy edge, under the scleral flap. The implant is secured in several places, as needed, and trimmed to fit the surgical site. The implant extends several millimeters posteriorly and to the sides of the sclerectomy site. Then, the scleral flap is secured to the implant using, for example, non-absorbable sutures. The tightness of the sutures is adjusted to prevent overflow and ocular hypotony, but to allow for a reasonable amount of aqueous to escape from the anterior chamber of the eye (filtration) in an amount sufficient to control the eye pressure. Finally, the conjunctiva and tenon are closed, and antibiotics and steroids are injected under the conjunctiva.

### BRIEF DESCRIPTION OF THE DRAWINGS

FIGS. 1a through 1d are top views (surgeon's views) of the major eye components and diagrammatically illustrate the steps associated with the positioning of the implant during a guarded filtration procedure.

FIG. 2 is a vertical cross-sectional side view of the eye, illustrating an implant made of a silicone sheet material in place following the guarded filtration procedure.

### DETAILED DESCRIPTION OF THE INVENTION

As noted above, in accordance with the invention, an implantable-grade sheet material is implanted so that it is positioned centrally, along the posterior edge of the sclerectomy site, and extends laterally on both sides over the sclera. The geometry of the implant is fashioned to allow it to conform to the globe of the eye, and to remain affixed to the tissue of the eye (sclera).

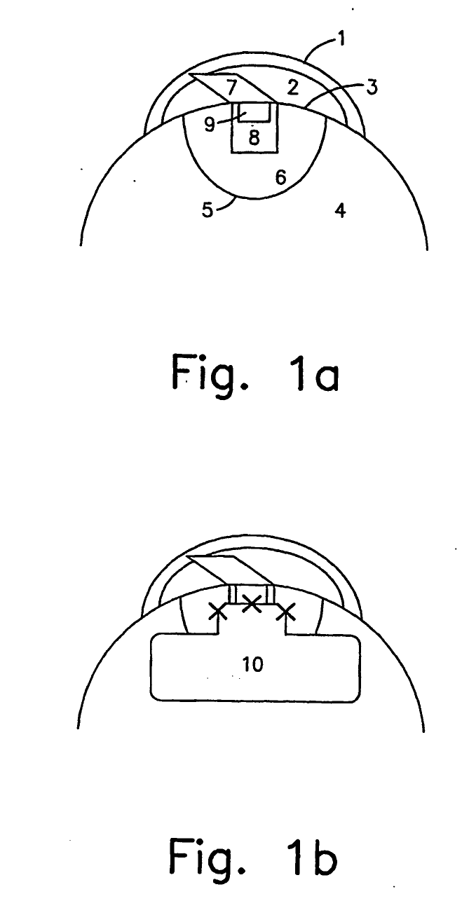

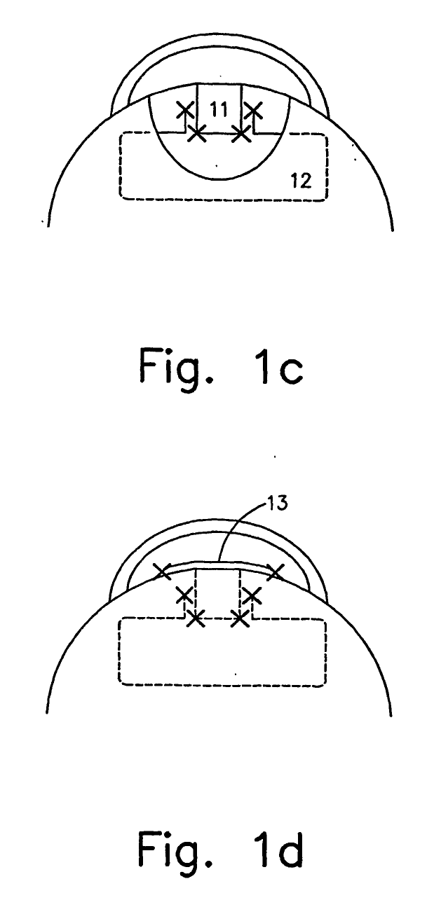

One preferred embodiment of the implant, and a method for positioning it on the eye, is illustrated in FIGS. 1a through 1d. The implant 10 is best seen in FIG. 1b, which illustrates an implant after it has been trimmed by the surgeon to fit the surgical site. In this embodiment, the implant has a rectangular base, preferably about 5 mm×10 mm in size, and an anterior extension of approximately 3 mm×5 mm. Of course, the actual dimensions and geometry of the implant sheet 10 will depend on the specific patient including, but not limited to, such factors as the surgical site condition, the amount of scar-free tissues available, and the severity of the glaucoma. The latter is a factor because the size of the implant will determine the size of the aqueous bleb (which contains the aqueous outflow) formed under the conjunctiva and Tenon's capsule (the outermost layers of the eye), thus determining the amount of filtration and the resulting intra-ocular pressure. Therefore, the implant could be trimmed by the surgeon into almost any shape that would achieve the desired effect.

The thickness of the implant is less than 100μ, preferably 25-50μ. The implant should thus be thin enough not to cause an elevated mass under the conjunctiva and Tenon's capsule, but still strong enough to withstand suturing through it without tearing. The “implantable-grade” (i.e. safe and tolerable to the eye tissues) material, such as silicone, for example, is highly flexible due to its thinness, yet equally strong. In a preferred embodiment, the implant is manufactured pre-molded to conform to the average eye globe. For example, the average eye has a diameter of 22 to 24 mm, therefore the radius of curvature of the implant is preferably about 11-14 mm. Other radii of curvature can be manufactured to fit different globes.

Silicone or methylmetacrylate are preferred as possible implant materials because they have been in long-term, widespread use as materials for various types of implants in and around the eye. For example, silicone and acrylic intraocular lens implants to replace the removed cataractous lenses in modern cataract surgery have been in use for many years with excellent safety and tolerability records. Similarly, silicone has been widely used over the years in tube shunts and valves for glaucoma surgery, in periocular bands for retinal detachment surgery, and in orbital fracture bone replacements. However, this invention also contemplates the use of any other biocompatible materials to which tissues do not adhere, and which are equally safe and tolerable for use in the invention. Many of such related materials are also demonstrating excellent safety records as intraocular and periocular implants.

As mentioned above, FIGS. 1a through 1d depict a preferred embodiment of the implant and procedure. In FIG. 1a, following local anesthesia, the Tenon's capsule and conjunctiva 4, covering the sclera 6, are cut from the limbus 3 and retracted backward, to create a fornix-based flap 5 (the fornix forms the cul-de-sac of the conjunctiva, under the lid; the limbus forms the border between the cornea and where the white of the eye begins). GFP can also be performed using a limbus-based flap, where Tenon's capsule and conjunctiva are severed at the upper fornix and dissected and retracted forward until the limbus is reached. The fornix-based Tenon's capsule and conjunctiva flap cannot be seen in FIGS. 1a through 1c, as it is pulled back toward the reader, therefore only the space under the flap, whose border is depicted at 5, revealing the exposed sclera 6, is drawn in FIGS. 1a through 1c for simplicity.

A partial-thickness limbus-based scleral flap 7 (partial thickness refers to a flap that is dissected, for example, two thirds of the way into the sclera, therefore one third of the sclera remains in the flap bed 8) is dissected in the exposed sclera 6 at the limbus 3. The scleral flap is then elevated and pulled forward toward the cornea 1 to expose the scleral bed 8. A sclerectomy (trabeculectomy) is then performed where part of the eye wall is removed in the scleral bed 8, resulting in a “window” or fistula 9 into the anterior chamber of the eye 2. At this point, the aqueous can drain from the anterior chamber of the eye through the sclerectomy, thus lowering the eye pressure.

Referring to FIG. 1b, a thin sheet of an implantable-grade implant 10, preferably, between 25 and 50 μm, is secured at the locations indicated by “X”, centrally at the posterior edge of the scierectomy site, so as not to obstruct the fistula, and lateral to the scleral bed on both sides. As stated earlier, the implant's final size is dependent upon such factors including, but not limited to, the size of the eye, the surgical site conditions, and the amount of scar-free and healthy tissues available. Additionally, the implant has a radius of curvature conforming to the contours of the eye globe. At this stage, the aqueous can still drain from the anterior chamber without resistance.

As shown in FIG. 1c, next the scleral flap is laid down as depicted at 11 to cover the fistula and part of the implant. The scleral flap is secured loosely enough to allow reasonable flow of aqueous from the anterior chamber through the sclerectomy, allowing the relief of excessive intra-ocular pressure. The sutures are nevertheless tight enough to prevent hypotony. The manner of securing the implant, along with the number of sutures used, depends on the degree of filtration and intra-ocular pressure desired. In addition, the posterior and lateral portions of the implant are tucked under the conjunctiva and Tenon's capsule layers, as shown by the dashed lines at 12. Usually, no additional sutures are necessary to secure the implant in those areas. After this point, the implant will prevent scarring and adherence of the scleral flap to the scleral bed, posterior and posterolateral to the sclerectomy site, and will prevent scarring and adherence between the Tenon's capsule and the episclera, the most common cause of failure of filtration with time. As a consequence, filtration will be maintained without the use of complication-causing, anti-scarring antimetabolites such as mitomycin C and 5FU, which are currently in use to prevent scarring.

Finally, as shown in FIG. 1d, the Tenon's capsule and conjunctiva are laid back down so as to completely cover the surgical site, and sutured back to the limbal cornea as shown at 13. The hatched Xs are the covered sutures of the scleral flap and implant which were shown in FIG. 1c. The final sutures are done so that fluid is unable to escape to the “outside world”, thus, rendering the surgical site “water-tight, and restoring the external anatomy of the eye.

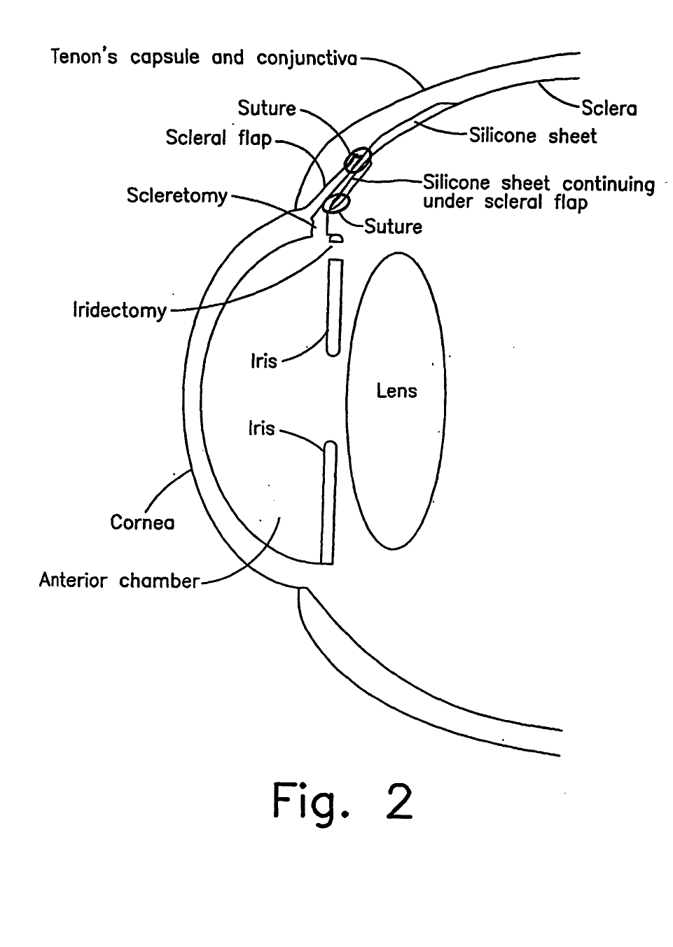

FIG. 2 is a vertical, cross-sectional view of the anterior eye, through the surgical site, illustrating the implant in one preferred embodiment as a silicone sheet under the conjunctiva and Tenon's capsule layers, inside the sclera and scleral flap. The scierectomy, as shown, allows the aqueous from the anterior chamber to drain under the scleral flap to the space under the Tenon's capsule and conjunctiva layers, which then forms a fluid-filled “bleb” containing the excess aqueous. The implant prevents the tissues from adhering to one another. Optionally, an iridectomy can be done to prevent the iris from adhering to the sclerectomy and obstructing drainage.

### EXAMPLE

### Animal Studies

In a recent rabbit study of the GFP (trabeculectomy), GFPs were performed in 14 nonglaucomatous eyes of 7 albino rabbits. In all 7, the right (study) eye underwent a GFP with a 100μ thick, implantable-grade implant made of a silicone sheet that extended from the posterior sclerectomy edge under the scleral flap, to several millimeters posterior and lateral to the scleral flap, under the conjunctiva and Tenon's capsule. GFPs were performed without a silicone implant in all 7 left (control) eyes. During the post-operative follow-up, conjunctival hyperemia and chemosis, anterior chamber reaction and lacrimation were graded. At the end of the follow-up period, 14-91 days following surgery, intra-ocular pressure (IOP) was measured with a Tonopen and the rabbits were sacrificed.

All 7 (100%) study eyes (GFP, with silicone implant) demonstrated a bleb at the end of the follow-up period, compared to only 1 of 7 (14.3%) control (GFP alone) eyes (p=0.001). IOP was lower in the study eyes compared to control eyes (8.3±1.8 mmHg vs. 10.6±1.3 mmHg, p+0.047). Hyperemia and chemosis scores were lower in the study eyes compared to control eyes (hyperemia: 0.94±0.74 vs. 1.33±0.86, p=0.001; Chemosis: 0.561±0.50 vs. 0.77±0.57, p=0.004). Anterior chamber reaction and lacrimation scores were similar in study and control eyes. Thus, implant implantation increases GFP success rate in albino rabbits.

Although the invention has been described with reference to specific embodiments, the description is intended to be illustrative of the invention and is not intended to be limiting. Various modifications and applications may occur to those skilled in the art without departing from the true spirit and scope of the invention as defined in the appended claims.

1. An ocular implant comprising implantable-grade material that inhibits adhesion and scarring of eye tissue following a guarded filtration procedure.

2. The ocular implant according to claim 1 wherein the implantable-grade material is silicone.

3. The ocular implant according to claim 1 wherein the implantable-grade material is methylmetacrylate.

4. The ocular implant according to claim 1 wherein the implantable-grade material is any biocompatible material to which tissues do not adhere.

5. A kit comprising the ocular implant according to claim 1.

6. A method for inhibiting adhesion and scarring following a guarded filtration procedure comprising positioning an ocular implant made of implantable-grade material at the edge of the sclerectomy site, under and around the scleral flap, such that said implant extends laterally and posteriorly from the sclerectomy site.

7. The method according to claim 6 wherein the implantable-grade material is silicone.

8. The method according to claim 6 wherein the implantable-grade material is methylmetacrylate.

9. The method according to claim 6 wherein the implantable-grade material is any biocompatible material to which tissues do not adhere.
